(19) United States
(12) Patent Application Publication
(10) Pub. No.: US 2013/0158127 A1
(43) Pub. Date: Jun. 20, 2013

(54) TEA TREE OIL DERIVATIVES

(71) Applicant: Jean-Christophe Sergere, Hauterive (FR)

(72) Inventor: Jean-Christophe Sergere, Hauterive (FR)

(21) Appl. No.: 13/715,681

(22) Filed: Dec. 14, 2012

Related U.S. Application Data
(60) Provisional application No. 61/570,468, filed on Dec. 14, 2011.

(51) Int. Cl. A61K 31/045; A61K 31/015

(52) U.S. Cl. CPC A61K 31/045; A61K 31/015
USPC 514/729; 568/825; 568/827

(57) ABSTRACT

Some embodiments comprise tea tree oil derivatives, and compositions and uses of same, which have decreased cytotoxic and irritating characteristics.

### CROSS-REFERENCE TO RELATED APPLICATIONS

This application claims the benefit of U.S. Provisional Application Ser. No. 61/570,468 filed Dec. 14, 2011, which is hereby incorporated by reference in its entirety.

### TECHNICAL FIELD

The present disclosure relates to tea tree oil derivatives and related methods and uses of same.

### BACKGROUND

Tea tree oil (“TTO”) has been extracted from the leaves of Melaleuca alternifolia and used for its medicinal properties for many years. TTO has been used to fight against microbial infections (bacteria, fungi, viruses). TTO contains immunomodulators, antioxidants, and insect repellents. It has also been suggested that some compounds found in TTO might be of major interest in cancer treatment.

TTO is an oil that may include dozens of constituent compounds. There is ISO standard, ISO 4730 (2004) (hereby incorporated by reference in full), that provides weight percentage ranges of particular constituent compounds required for an oil to be considered TTO. There are also Australian Standards, as well as specifications in the German Drugs Code (DAC) regarding percentages of constituent compounds (all such standards and specifications also hereby incorporated by reference in full). According to the ISO standard, TTO includes, by weight, terpinen-4-ol in an amount of 30-48%; γ-terpinene in an amount of 10-28%; α-terpinene in an amount of 5-13% 1,8-Cineole in an amount of 0-15%; α-terpinolene in an amount of 1.5-5%; α-terpineol in an amount of 1.5-8%; α-pinene 1-6%, and p-cymene in an amount of 0.5-8%.

TTO includes several classes of constituent compounds, including monoterpenes, monoterpenic alcohols, sesquiterpenes, sesquiterpene alcohols, and aromatic compounds. Monoterpenes, including monoterpenic alcohols, generally account for about 80-90% of TTO by weight. Exemplary monoterpenic alcohols include terpinen-4-ol and α-terpineol. Exemplary monoterpenes include γ-terpinene, α-terpinene, 1,8 cineole, p-cymene, α-pinene, terpinolene, limonene and sabinene.

Sesquiterpenes, including sesquiterpenic alcohols, generally account for less than about 10% of TTO by weight. Exemplary sesquiterpenes include aromadendrene, d-cadinene, and ledene (viridiflorene). Exemplary sesquiterpenic alcohols include globulol and viridiflorol.

Many of the TTO constituent compounds of interest have been the mono- and sesquiterpenic alcohols. For example, a broad range of biological activity of terpinen-4-ol, a monoterpenic alcohol, has been documented. Terpinen-4-ol has exhibited antibacterial activity, antifungal activity, antiviral activity, antiprotozoal activity, acaricidal activity, suppression of superoxide production by monocytes (but not neutrophils), anti-inflammatory activity and potential anti-cancer activity. See, e.g., Loughlin R et al. (2008), “Comparison of the cidal activity of tea tree oil and terpinen-4-ol against clinical bacterial skin isolates and human fibroblast cells,” Lett. Appl. Microbiol. 46(4): 428-433; Budzynska A et al. (2011), “Antibiofilm Activity of Selected Plant Essential Oils and their Major Components,” Pol. J. Microbiol, 60(1) 35-41; Mondello F et al. (2006), “In vivo activity of terpinen-4-ol, the main bioactive component of Melaleuca alternifolia Cheel (tea tree) oil against azole-susceptible and—resistant human pathogenic Candida species,” BMC Infect. Dis., 3; 6: 158; Oliva B et al. (2003), “Antimycotic activity of Melaleuca alternifolia essential oil and its major components,” Lett. Appl. Microbiol. 37(2): 185-187; Garozzo A et al. (2009), “In vitro activity of Melaleuca alternifolia essential oil,” Lett. Appl. Microbiol., 49(6): 806-808; Mikus J. et al. (2000), “In vitro effect of essential oils and isolated mono- and sesquiterpenes on Leishmania major and Trypanosoma brucei,” Planta Med., 66(4): 366-368; Walton S F et al. (2004), “Acaricidal activity of Melaleuca alternifolia (tea tree) oil: in vitro sensitivity of Sarcoptes scabiei var hominis to terpinen-4-ol,” Arc. Dermatol., 140(5): 563-566; Budhiraja S S et al. (1999) J. Manipulative Physiol. Ther.; Brand C. et al. (2001), “The water-soluble components of the essential oil of Melaleuca alternifolia (tea tree oil) suppress the production of superoxide by human monocytes, but not neutrophils, activated in vitro,” Inflamm. Res., 50(4): 213-219; Hart P H et al. (2000), “Terpinen-4-ol, the main component of the essential oil of Melaleuca alternifolia (tea tree oil), suppresses inflammatory mediator production by activated human monocytes,” Inflamm. Res., 49(11): 619-626; Bozzuto G. et al. (2011), “Tea tree oil might combat melanoma,” Planta Med., 77(1): 54-56; Greay S J et al. (2010), “Induction of necrosis and cell cycle arrest in murine cancer cell lines by Melaleuca alternifolia (tea tree) oil and terpinen-4-ol,” Cancer Chemother. Pharmacol. 65(5): 877-888; and Calcabrini A. et al. (2004), “Terpinen-4-ol, the main component of Melaleuca alternifolia (tea tree) oil inhibits the in vitro growth of human melanoma cell,” J. Invest. Dermatol. 122(2): 349-360.

Similarly, α-terpineol, a monoterpenic alcohol, may suppress the production of superoxide by monocytes but not neutrophils, suggestive of anti-inflammation activity. See, e.g., Brand C. et al. (2001), “The water-soluble components of the essential oil of Melaleuca alternifolia (tea tree oil) suppress the production of superoxide by human monocytes, but not neutrophils, activated in vitro,” Inflamm. Res., 50(4): 213-219.

Additionally, viridiflorol, a sesquiterpenic alcohol, has exhibited antioxidant activity. See, e.g., Pino J A et al. (2010), “Phytochemical analysis and in vitro free-radical-scavenging activities of the essential oils from leaf and fruit Melaleuca leucadendra L,” Chem. Biodivers., 7(9): 2281-2288.

TTO compositions, however, may include constituent compounds that are less desired for medical applications. For example, certain compounds may have undesired toxicity or irritation properties. The less desired constituent compounds in TTO may include monoterpenes and their degradation products. Nonlimiting examples of such constituent compounds include γ-cymene, terpinolene, α-terpinene, γ-terpinene, δ-limonene, x-terpinene, and p-cymene.

Some of these monoterpenes with undesired properties are however well known to be active against microbes and parasites. This includes α-pinene, 1,8-cineole, α-terpinene, γ-terpinene, and p-cymene as strong antibacterial and antifungal molecules, and terpinolene as an antifungal molecule. Many scientific articles describe the antimicrobial properties of these monoterpenes, including: Pichette A. et al. (2006), Phytother. Res. 20(5): 371-373 ; Santoyo S. et al. (2005), J. Food Prot. 68(4): 790-795 ; Kim K J et al. (2003), Planta Med. 69(3): 274-277, Xia Z. et al. (1999), Human Yi Ke Da Xue Xue Bao 24(6) : 507-509 ; Hammer K A et al., J. Appl. Microbiol. 95(4): 853-860 ; Jiang Y. et al. (2011), Environ. Toxicol. Pharmacol. 32(1): 63-68, Santoyo S. et al. (2005), J. Food Prot. 68(4) : 790-795 ; Kim K J et al. (2003), Planta Med. 69(3): 274-277; Hammer K A et al., J. Appl. Microbiol. 95(4): 853-860; Morcia et al. (2012), Food Addit. Contam. Part A Chem. Anal. Control Expo. Risk Assess. 29(3): 415-422; Jiang Y. et al. (2011), Environ. Toxicol. Pharmacol. 32(1) 63-68 Hendry ER et al. (2009), J. Antimicrob. Chemother. 64(6): 1219-1225; Viljoen A. et al. (2003), J. Ethnopharmacol. 88(2-3): 137-143 ; Terzi V. et al. (2007), Lett. Appl. Microbiol. 44(6): 613-618 ; Faroog A. et al. (2002) ; Z. Naturforsch. C. 57(9-10): 863-866; Terzi V. et al. (2007); Lett. Appl. Microbiol. 44(6): 613-618; Piras A. et al. (2011), Nat. Prod. Commun. 6(10): 1523-1526; Cristani M (2007), J. Agric. Food Chem. 55(15): 6300-6308 ; Parveen M et al. (2004), J. Antimicrob. Chemother. 54(1): 46-55; Yucel N and Aslim B (2011), Foodborne Pathog. Dis. 8(1): 71-76; Sekine T. et al. (2007), J. Chem. Ecol. 33(11): 23-32; Cristani M. et al. (2007), J. Agric. Food Chem. 55(15): 6300-6308; Periago P. M. et al. (2004), J. Food Prot. 67(7): 402-416. These major monoterpenes (α-pinene, 1,8 cineole, terpinolene, γ-terpinene, α-terpinene and p-cymene) represent more than 40% of a standard TTO, but less than 4% in FJ1 (an exemplary TTO derivative described herein and comprising some embodiments). α-pinene is decreased more than one hundred times, terpinolene more than 5 times, 1,8 cineole more than 10 times, α-terpinene more than 37 times, γ-terpinene 10 times, and limonene more than 26 times, in FJ1 compared to TTO. Thus, one could expect that depletion of these molecules from ISO TTO, representing more than 40% of total amount of ISO TTO, should be correlated with a decrease in antibacterial and antifungal activities.

### BRIEF DESCRIPTION OF THE DRAWINGS

Referring now to the drawings, preferred illustrative embodiments are shown in detail. Although the drawings represent some embodiments, the drawings are not necessarily to scale and certain features may be exaggerated, removed, or partially sectioned to better illustrate and explain the present inventive concepts. Further, the embodiments set forth herein are not intended to be exhaustive or otherwise limit or restrict the claims to the precise forms and configurations shown in the drawings and disclosed in the following detailed description. In addition, where the drawings show calculations, graphs, or data plots, such graphical representations are simply illustrative of the present inventive concepts and not intended to be exact representations.

FIG. 1a shows the compared cytotoxicity of TTO and an exemplary TTO derivative solubilized at 10% in 10% DMSO.

FIG. 1b shows the compared cytotoxicity of TTO and an exemplary TTO derivative solubilized at 10% in 6% Tween 60.

FIG. 2 shows Dengue viral genome equivalents per ml of serum each day post-challenge plotted of arithmetic (upper panel) and logarithmic (lower panel) scales.

FIG. 3 is an image showing the effect of FJ1 on DENV infectivity by immunofluorescence assay.

### DETAILED DESCRIPTION

Standard TTO, such as ISO TTO, is known to have antimicrobial activity against species such as certain bacteria, fungae, and viruses. Many studies describe monoterpenes as a strong active antimicrobial components in such TTO. At the same time, many monoterpenes are believed to have potential toxicity, with indications that they should not be ingested, or if so, only in very limited amounts. Should one wish to reduce monoterpene content in TTO in order to reduce untoward effects, it might be expected that such a reduction would result in decreased antimicrobial activity.

Contrary to this conventional view, the inventor has discovered unexpectedly that decreasing monoterpene content in some embodiments results in TTO derivative(s) which maintain, or even increase, antimicrobial activity. In addition, the inventor has discovered unexpectedly that, contrary to conventional wisdom, some embodiments comprising such TTO derivative(s), enriched in components such as terpene-4-ol, were less cytotoxic and irritating than formulations such as ISO TTO. Thus, the inventor has discovered unexpectedly some embodiments comprising TTO derivatives, and uses of same, which retain or have improved antimicrobial activity, while being less cytotoxic and irritating.

Using fractionation techniques, the inventor has synthesized useful TTO derivatives. Exemplary TTO derivatives may maximize TTO constituent compounds having desired biological activity such as, by way of non-limiting example, monoterpenic alcohols, sesquiterpenes, and sesquiterpenic alcohols. Exemplary TTO derivatives may minimize, substantially eliminate, or completely eliminate TTO constituent compounds having undesired toxicity and/or irritation properties such as, by way of non-limiting example, monoterpenes other than monoterpenic alcohols. Exemplary TTO derivatives may both maximize TTO constituent compounds having desired biological activity and minimize, substantially eliminate, or completely eliminate TTO constituent compounds having undesired toxicity and/or irritation properties.

Throughout this disclosure, many numerical ranges are disclosed to indicate content percentages of particular constituent compounds in TTO derivatives. Unless stated otherwise, all such ranges are by weight, with the entire weight of the TTO derivative as 100%. Additionally, when multiple nested ranges are identified for a particular constituent compound, any of the disclosed lower limits may be used in connection with any of the disclosed lower limits for the particular constituent compound.

### Enriching TTO Constituents Having Desired Biological Activity

Monoterpenic alcohols, as noted above, are understood to have beneficial biological activity. In some embodiments, without limitation to only specifically disclosed embodiments and without disclaimer or waiver of other embodiments or subject matter, TTO derivatives are contemplated that increase the weight percent of such constituents relative to TTO. For instance, terpinen-4-ol may range from 30-48% by weight according to the ISO standard. It is contemplated that exemplary TTO derivatives may include terpinen-4-ol higher than 48% by weight. It is contemplated that TTO derivatives may contain terpinen-4-ol in weight percentages ranging from, for example, 50 to 85%, 55 to 80%, or 60 to 75% by weight.

Similarly, the monoterpenic alcohol, α-terpineol, may range from 1.5 to 8% in TTO. Exemplary TTO derivatives may include α-terpineol in the upper half of that range and higher than that range. It is contemplated that TTO derivatives may contain include α-terpineol in weight percentages ranging from, for example, 2 to 12%, 3 to 10%, or 4 to 9% by weight.

Sesquiterpenes, as noted above, are understood to have beneficial biological activity. Thus, TTO derivatives are contemplated that increase the weight percent of such constituents relative to TTO. For instance, aromadendrene may range from trace-3% by weight according to the ISO standard. Exemplary TTO derivatives may include aromadendrene in the upper half of that range and higher than that range. It is contemplated that TTO derivatives may contain aromadendrene in weight percentages ranging from, for example, 0.5 to 6%, 1 to 5%, or 1.5 to 4% by weight.

Sesquiterpenic alcohols, as noted above, are understood to have beneficial biological activity. Thus, TTO derivatives are contemplated that increase the weight percent of such constituents relative to TTO. For instance, globulol may range from trace-1% by weight according to the ISO standard. Exemplary TTO derivatives may include globulol in the upper two-thirds of that range and higher than that range. It is contemplated that TTO derivatives may contain globulol in weight percentages ranging from, for example, 0.1 to 3%, 0.2 to 2%, or 0.3 to 1% by weight.

Similarly, the sesquiterpenic alcohol, viridiflorol, may range from trace to 1% by weight in TTO. Exemplary TTO derivatives may include viridiflorol in the upper 90% of that range and higher than that range. It is contemplated that TTO derivatives may contain include viridiflorol in weight percentages ranging from, for example, zero to 3%, trace to 2%, or 0.1 to 1% by weight.

Depleting TTO Constituents Having Toxic and/or Irritation Effects

As noted above, certain constituents in TTO have toxic and/or irritation effects. In some embodiments, without limitation to only specifically disclosed embodiments and without disclaimer or waiver of other embodiments or subject matter, it is contemplated to eliminate, substantially eliminate or simply minimize such constituents, including monoterpenes. As such, TTO derivatives are contemplated that decrease the weight percent of such constituents relative to TTO. For instance, α-pinene may range from 1-6% by weight according to the ISO standard. It is contemplated that exemplary TTO derivatives may include α-pinene less than 1% by weight. It is contemplated that TTO derivatives may contain α-pinene in weight percentages ranging from, for example, zero to 1%, trace to 0.5%, or trace to 0.1% by weight. In defining ranges, it is contemplated that any of the disclosed lower limits may be used in connection with any of the disclosed upper limits.

Similarly, the monoterpene, terpinolene, may range from 1.5 to 5% in TTO. Exemplary TTO derivatives may include terpinolene in the lower half of that range and below than that range. It is contemplated that TTO derivatives may contain include terpinolene in weight percentages ranging from, for example, zero to 3%, trace to 2%, or 0.02 to 0.8% by weight.

The monoterpene, 1,8-cineole, may range from trace to 15% in TTO. Exemplary TTO derivatives may include 1,8-cineole in the lower half of that range and below than that range. It is contemplated that TTO derivatives may contain include 1,8-cineole in weight percentages ranging from, for example, zero to 6%, trace to 3%, or 0.01 to 0.4% by weight.

The monoterpene, α-terpinene, may range from 5 to 13% in TTO. Exemplary TTO derivatives may include α-terpinene in the lower half of that range and below than that range. It is contemplated that TTO derivatives may contain include α-terpinene in weight percentages ranging from, for example, zero to 9%, trace to 5%, or 0.1 to 0.5% by weight.

The monoterpene, γ-terpinene, may range from 10 to 28% in TTO. Exemplary TTO derivatives may include γ-terpinene in the lower half of that range and below than that range. It is contemplated that TTO derivatives may contain include γ-terpinene in weight percentages ranging from, for example, zero to 18%, trace to 9%, or 0.8 to 3% by weight.

The monoterpene, p-cymene, may range from 0.5 to 8% in TTO. Exemplary TTO derivatives may include p-cymene in the lower half of that range and below than that range. It is contemplated that TTO derivatives may contain include p-cymene in weight percentages ranging from, for example, zero to 4%, trace to 2%, or 0.1 to 0.5% by weight.

The monoterpene, limonene, may range from 0.5 to 1.5% in TTO. Exemplary TTO derivatives may include limonene in the lower half of that range and below than that range. It is contemplated that TTO derivatives may contain include limonene in weight percentages ranging from, for example, zero to .7%, trace to .5%, or 0.02 to 0.1% by weight.

The monoterpene, sabinene, may range from trace to 3.5% in TTO. Exemplary TTO derivatives may include sabinene in the lower half of that range and below than that range. It is contemplated that TTO derivatives may contain include sabinene in weight percentages ranging from, for example, zero to 1.6%, trace to 1%, or trace to 0.1% by weight.

Collectively, exemplary TTO derivatives may contain less than 5% of the major monoterpenes present in standard TTO (ISO 4730-2004).

### Synthesizing Exemplary TTO Derivatives

In some embodiments, and as one example only, ISO TTO was loaded in a heated balloon and distillated in a 2.5 meter column under relative vacuum (0 to 1 millibar) at low and increasing temperature (under 70° C., preferably 60° C.) with a back loop of condensation. Distillation was carried out until monoterpenes reach less than 4% of the total amount in the heated balloon.

### Testing for Cytotoxicity of Exemplary TTO Derivatives

There are many methods that may be used to evaluate the cytotoxicity of exemplary TTO derivatives. By way of non-limiting example, the cytotoxicity of ISO TTO versus exemplary TTO derivatives may be evaluated by measuring cell survival of a human foreskin fibroblasts (HFF) primary cell line in the absence or presence of decreasing concentrations of the extracts. Cell survival may be monitored by dosage of total proteins. The effect of ISO TTO and certain TTO derivatives on human primary fibroblasts grown in vitro was investigated according to the methods of Papazisis KT et al. (1997), “Optimization of the sulforhodamine B colorimetric assay,” J. Immunol. Methods, 208: 151-8. Adherent HFF (Human Foreskin Fibroblasts) were cultured in 25 cm² dishes in supplemented MEM (Minimum Essential Medium, Life Technologies Gibco), in a chamber at 37° C. and 5% CO₂. 500 mL of MEM was supplemented with fetal calf serum (10% final), glutamine 2 mM (5 mL), antibiotics (5 mL penicillin/streptomycin i.e. 0.1 mU/mL, 0.5 mL ampicillin i.e. 10 μg/mL, 0.25 mL gentamicin i.e. 25 μg/mL) and fungicides (5 mL fungizone i.e. 2.5 μg/mL). Adherent HFF cells at logarithmic growth phase were plated (200 μL per well) in 96-well flat bottom microplates at densities of 100.000 cells per well. Microplates were left for 24 hours at 37° C. in MEM-S medium. Cells were then left with decreasing amounts of TTO or TTO derivatives for 24 additional hours, in parallel of a negative control (without TTO or TTO derivatives) and a positive control (10% DMSO). Each experiment was performed in triplicates. After 24 hours, TriChloroacetic Acid (TCA) 50%was added to the top of 200μL culture medium in each well to produce a final TCA concentration of 10%. The microplate was left for 2 hours at 4° C. and subsequently washed 3 times with deionized water. Microplates were then left to dry air room temperature. 100 μL of 0.4% (w/v) sulforhodamine B (SRB) in 1% acetic acid solution were added to each well and left at room temperature for 20 minutes. SRB was then washed 3 times with 1% acetic acid before air drying. Bound SRB was solubilised with 200 μL 10 mM unbuffered Tris-Base solution and plates were left on a plate shaker for at least 5 minutes. Absorbance at 550 nm was then read in a 96-well plate reader. Collected measures were then treated and the absorbance of each mean value of a triplicate was reported on a graph, with the different concentrations tested in x-coordinates, and the absorbance (directly reflecting cell survival) in γ-coordinates.

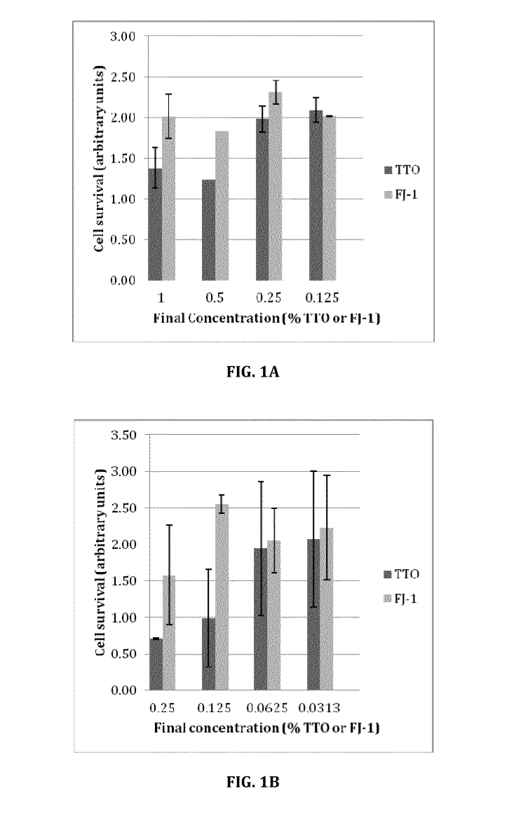

FIGS. 1A and 1B show the cell survival of HFF cells in the presence of decreasing amounts of TTO or the TTO derivative “FJ1” respectively solubilized in DMSO (FIG. 1A) or Tween 60 (FIG. 1B). The x-coordinate of each couple of bars indicates the final concentration in TTO or TTO derivative. The cell survival of TTO and TTO derivative (FJ1) are respectively in dark grey and light grey. Standard deviations of the triplicates are reported on the bars. For concentrations up to 0.5% (DMSO as a solvent, FIG. 1A) or 0.125% (Tween 60 as a solvent, FIG. 1B), there is a significant difference in cell survival between TTO and TTO derivative.Terpinen-4-ol is known to be an antiproliferative molecule, and an anti-cancer candidate (see for example, Greasy S J et al. (2010), Cancer Chemother Pharmacol 65(5): 877-888). Thus it could be expected that an enrichment of terpinen-4-ol in FJ1 compared to ISO TTO could decrease the survival of cells in cytotoxicity assays, leading to a decrease in the monitoring of total proteins by colorimetric assays using sulforhodamine B. Unexpectedly, in our work, total proteins and thus cell survival were increased, demonstrating that the TTO derivative FJ1 is less cytotoxic than ISO TTO alone. This experiment has been performed 3 times.

### Testing for Desired Biological Activity of Exemplary TTO Derivatives

There are many methods that may be used to evaluate desired biological activity of exemplary TTO derivatives. By way of non-limiting example, the antibacterial and antifungal properties of ISO TTO versus exemplary TTO derivatives may be evaluated by comparing their Minimal Inhibitory Concentration (MIC) on numerous bacterial strains and fungal strains according to international protocols. Each bacterial strain at logarithmic growth phase was plated at 100 000 cfu/mL in a 96 well round bottom microplate in growth medium, in the presence of serial dilutions of ISO TTO or TTO derivatives. Growth controls (without ISO TTO or TTO derivatives) and background controls (without bacteria or fungi) were also performed. Microplates were then incubated in standard growth conditions. After growth of the positive control (bacteria without ISO TTO or TTO derivative), the MIC (Minimal Inhibitory Concentration) was read by eye as the lowest concentration of ISO TTO or TTO derivative at witch there was no visible growth.

### Exemplary Methods of Use and Treatment

Exemplary TTO derivatives can be incorporated at 1.25% final concentration in intimate hygiene products (soap, gel, vaginal capsules, vaginal creams) to prevent urinary tract infections, or prevent or cure vaginosis.

Exemplary TTO derivatives can be incorporated at 1.25% final concentration in anti-acne compositions (as nonlimiting examples, lotion, soap, cream) to prevent or cure acne.

Exemplary TTO derivatives can be incorporated at 1.25% final concentration in oral care products (toothpaste, gum, mouthwash) to prevent or cure mouth infections, candidosis, caries, gingivitis, periodontis).

Exemplary TTO derivatives can be incorporated at 0.31% final concentration as a preservative for ingredient preservation, food, or cosmetics.

Exemplary TTO derivatives can be incorporated at 0.16% to 2.5% final concentration in solutions for disinfection (gel, spray), the lower concentrations being efficient against Salmonella, Yersinia, Bacillus strains, the higher concentrations being efficient against Vibrio and Legionella. This also includes hygiene compositions such as antibacterial and antifungal compositions for hygiene such as creams and balms for podologists.

Exemplary embodiments may comprise an alcohol gel antiseptic of the following formula, or variations thereof:

| INGREDIENT | % |
|---|---|
| ALCOHOL | 68 |
| WATER | 30.3-28.8 |
| TTO DERIVATIVE (FJ-1) | 1-2 |
| GELIFIANT | 0.5 |
| THYME ESSENTIAL OIL | 0.1-0.5 |
| RAVINTSARA ESSENTIAL OIL | 0.1-0.2 |

Exemplary embodiments may comprise an exfoliating and antiseptic foot balm of the following formula, or variations thereof:

| INGREDIENT | % |
|---|---|
| WATER FLOWER | 48.5-58 |
| SESAMUM SEED OIL | 15 |
| WATER | 15-5 |
| GLYCERIN | 5 |
| AUTOEMULSIONANT BASE | 5 |
| MINERAL EXFOLIANT | 8 |
| KAOLIN | 2 |
| TTO DERIVATIVE (FJ-1) | 1-1.5 |
| GELLING AGENT | 0.3 |
| PRESERVATIVES | 0.2 |

Exemplary embodiments may comprise an antiseptic massage oil of the following formula, or variations thereof:

| INGREDIENT | % |
|---|---|
| CAPRYLCAPRYLIC ACID | 76.45 |
| SESAMUM INDICUM OIL | 23.25-21.55 |
| TTO DERIVATIVE (FJ-1) | 0.75-2 |

### EXAMPLES

The following examples are provided without limiting the invention to only those embodiments described herein and without disclaiming other embodiments or subject matter.

### Example 1

### Preparation of 4 Batches of Exemplary TTO Derivatives

Each batch of TTO derivative was analyzed by GC/FIP (Gas Chromatography coupled to a Flame Ionization Detector). Qualitative and quantitative data collected are reported in the following table, listing the major constituent compounds and their relative amounts. The table summarizes representative results collected from four independent batches. The mean value is also reported.

| Constituent | | | | | |
|---|---|---|---|---|---|
| Compound | Batch 1 | Batch 2 | Batch 3 | Batch 4 | Mean |
| α-pinene | — | — | — | — | — |
| Terpinolene | 0.642 | 0.786 | 0.478 | 0.610 | 0.629 |
| 1,8-cineole | 0.096 | 0.249 | — | 0.125 | 0.118 |
| (=eucalyptol) | | | | | |
| α-terpinene | 0.225 | 0.245 | 0.239 | 0.325 | 0.259 |
| γ-terpinene | 1.719 | 2.706 | 1.625 | 2.418 | 2.117 |
| p-cymene | 0.115 | 0.391 | 0.187 | 0.198 | 0.223 |
| Limonene | 0.027 | 0.064 | 0.055 | 0.066 | 0.053 |
| Sabinene | — | — | — | — | — |
| terpinen-4-ol | 70.345 | 72.516 | 71.037 | 74.348 | 72.062 |
| α-terpinol | 4.367 | 6.177 | 8.021 | 7.771 | 6.584 |
| Aromadendrene | 3.279 | 1.892 | 2.468 | 2.209 | 2.462 |
| d-cadinene | 2.123 | 1.556 | 0.555 | 1.285 | 1.380 |
| Iedene | 2.703 | 2.293 | 0.890 | 1.652 | 1.885 |
| (=viridiflorene) | | | | | |
| Globulol | 0.335 | 0.576 | 0.854 | 0.417 | 0.546 |
| Viridiflorol | 0.116 | 0.296 | 0.361 | 0.178 | 0.238 |

### Example 2

### Preparation of 3 Batches of ISO TTO

Each batch of commercial ISO TTO was analyzed by GC/FIP (Gas Chromatography coupled to a Flame Ionization Detector). Qualitative and quantitative data collected are reported in the following table, listing the major constituent compounds and their relative amounts. The table summarizes representative results collected from three independent batches. The mean value is also reported.

| Constituent | | | | |
|---|---|---|---|---|
| Compound | Batch 1 | Batch 2 | Batch 3 | Mean |
| α-pinene | 2.332 | 2.930 | 2.736 | 2.666 |
| terpinolene | 3.338 | 3.364 | 3.260 | 3.32 |
| 1,8-cineole | — | — | 1.701 | 0.567 |
| (=eucalyptol) | | | | |
| α-terpinene | 8.913 | 9.756 | 10.570 | 9.746 |
| γ-terpinene | 19.522 | 21.780 | 22.254 | 21.185 |
| p-cymene | — | — | 1.838 | 0.613 |
| limonene | 1.008 | 1.667 | 1.552 | 1.409 |
| sabinene | 0.171 | 0.103 | 0.134 | 0.136 |
| terpinen-4-ol | 45.515 | 42.729 | 41.809 | 43.351 |
| α-terpinol | 3.297 | 4.156 | 4.094 | 3.849 |
| aromadendrene | — | — | 1.047 | 0.389 |
| d-cadinene | — | — | 0.638 | 0.213 |
| Iedene | — | — | 0.754 | 0.251 |
| (=viridiflorene) | | | | |
| globulol | — | — | 0.358 | 0.119 |
| viridiflorol | — | — | 0.157 | 0.052 |

### Example 3

### Constituent Comparison ISO TTO to Exemplary TTO Derivative

The mean weight percentages of the constituent compounds from the exemplary TTO derivatives were compared to the mean weight percentages of the constituent compounds from the ISO-compliant TTO batches. The ISO TTO batches served as a baseline in the percentage increases and decreases.

| | Mean | Mean | | |
|---|---|---|---|---|
| Constituent | TTO | ISO | Percentage | Percentage |
| Compound | Derivative | TTO | Increase | Decrease |
| α-pinene | — | 2.667 | | 99% |
| terpinolene | 0.629 | 3.320 | | 81% |
| 1,8-cineole | 0.118 | 0.567 | | 79% |
| (=eucalyptol) | | | | |
| α-terpinene | 0.259 | 9.746 | | 97% |
| γ-terpinene | 2.117 | 21.185 | | 90% |
| p-cymene | 0.223 | 0.613 | | 63% |
| limonene | 0.053 | 1.409 | | 97% |
| sabinene | — | 0.136 | | 99% |
| terpinen-4-ol | 72.062 | 43.351 | 66% | |
| α-terpinol | 6.584 | 3.849 | 71% | |
| aromadendrene | 2.462 | 0.389 | 533% | |
| d-cadinene | 1.380 | 0.213 | 547% | |
| ledene | 1.885 | 0.251 | 651% | |
| (=viridiflorene) | | | | |
| globulol | 0.546 | 0.119 | 358% | |
| viridiflorol | 0.238 | 0.052 | 357% | |

### Example 4

### Reduced Cytotoxicity of Exemplary TTO Derivative Relative to ISO TTO

The cytotoxicity of TTO and exemplary TTO derivative is evaluated by measuring cell survival of a human foreskin fibroblasts (HFF) primary cell line in the H absence or presence of decreasing concentrations of the extracts. Cell survival is monitored by dosage of total proteins.

Adherent HFF (Human Foreskin Fibroblasts) were cultured in 25 cm² dishes in supplemented MEM (Minimum Essential Medium, Life Technologies Gibco), in a chamber at 37° C. and 5% CO₂. 500 mL of MEM was supplemented with fetal calf serum (10% final), glutamine 2 mM (5 mL), antibiotics (5 mL penicillin/streptomycin i.e. 0.1 mU/mL, 0.5 mL ampicillin i.e. 10 μg/mL, 0.25 mL gentamicin i.e. 25 μg/mL) and fungicides (5 mL fungizone i.e. 2.5 μg/mL). Adherent HFF cells at logarithmic growth phase were plated (200 μL per well) in 96-well flat bottom microplates at densities of 100.000 cells per well. Microplates were left for 24 hours at 37° C. in MEM-S medium. Cells were then left with decreasing amounts of TTO or TTO derivatives for 24 additional hours, in parallel of a negative control (without TTO or TTO derivatives) and a positive control (10% DMSO). Each experiment was performed in triplicates. After 24 hours, TriChloroacetic Acid (TCA) 50%was added to the top of 200μL culture medium in each well to produce a final TCA concentration of 10%. The microplate was left for 2 hours at 4° C. and subsequently washed 3 times with deionized water. Microplates were then left to dry air room temperature. 100μL of 0.4% (w/v) sulforhodamine B (SRB) in 1% acetic acid solution were added to each well and left at room temperature for 20 minutes. SRB was then washed 3 times with 1% acetic acid before air drying. Bound SRB was solubilised with 200 μL 10 mM unbuffered Tris-Base solution and plates were left on a plate shaker for at least 5 minutes. Absorbance at 550 nm was then read in a 96-well plate reader. Collected measures were then treated and the absorbance of each mean value of a triplicate was reported on a graph, with the different concentrations tested in x-coordinates, and the absorbance (directly reflecting cell survival) in γ-coordinates.

Batch number 1 of ISO TTO and batch number 1 of exemplary TTO derivative were solubilized at a 10% concentration in 10% dimethylsulfoxide (DMSO) and were tested for their cytotoxicity.

Next, batch number 1of ISO TTO and batch number 1 of exemplary TTO derivatives were solubilized in 6% Tween 60 and were tested for their cytotoxicity.

FIGS. 1A and 1B show the cell survival of HFF cells in the presence of decreasing amounts of TTO or the TTO derivative FJ1 respectively solubilized in DMSO (FIG. 1A) or Tween 60 (FIG. 1 B). The x-coordinate of each couple of bars indicates the final concentration in TTO or TTO derivative. The average cell survival triplicates of TTO and TTO derivative (FJ1) are respectively in dark grey and light grey. Standard deviations of the triplicates are also reported on the bars. It clearly appears that for concentrations superior or equal to 0.5% (DMSO as a solvent, FIG. 1A) or 0.125% (Tween 60 as a solvent, FIG. 1B), there is a significant difference in cell survival between TTO and TTO derivative.

Terpinen-4-ol is known to be an antiproliferative molecule, and an anti-cancer candidate (see for example, Greasy S J et al. (2010), Cancer Chemother Pharmacol 65(5): 877-888). Thus it could be expected that an enrichment of terpinen-4-ol in FJ1 compared to ISO TTO could decrease the survival of cells in cytotoxicity assays, leading to a decrease in the monitoring of total proteins by colorimetric assays using sulforhodamine B. Unexpectedly, total proteins and thus cell survival were increased, demonstrating that the TTO derivative FJ1 is less cytotoxic than ISO TTO alone. This experiment has been performed 3 times.

### Example 5

### Increased Antimicrobial Activity of Exemplary TTO Derivatives Relative to ISO TTO

Batches 1-3 of ISO TTO and batches 1-4 of exemplary TTO derivatives were compared for their respective antimicrobial activities by evaluating their Minimal Inhibitory Concentration (MIC) on 19 bacterial strains and 3 fungal strains.

Each bacterial strain at logarithmic growth phase was plated at 100 000 cfu/mL in a 96 well round bottom microplate in growth medium, in the presence of serial dilutions of ISO TTO or TTO derivatives. Growth controls (without ISO TTO or TTO derivatives) and background controls (without bacteria or fungi) were also performed. Microplates were then incubated in standard growth conditions. After growth of the positive control (bacteria without ISO TTO or TTO derivative), the MIC (Minimal Inhibitory Concentration) was read by eye as the lowest concentration of ISO TTO or TTO derivative at witch there was no visible growth.

The results of batch number 1 of ISO TTD and batch number 1 of Exemplary TTO derivative are indicated below (similar results were obtained for other batches):

| | | Exemplary |
|---|---|---|
| | | TTO |
| Strain | ISO TTO | Derivative |
| Actinomyces naeslundii | 2.5% | 1.25% |
| Bacillus atrophaeus | 0.31% | 0.16% |
| Bacillus cereus | 0.31% | 0.31% |
| Clostridium perfringens | — | — |
| Escherichia coli | 0.31% | 0.16% |
| Fusobacterium nucleatum | 0.31% | 0.635% |
| Legionella pneumophila | — | 1.25% |
| Listeria monocytogenes | 0.635% | 0.31% |
| Propionibacterium acnes | 2.50% | 1.25% |
| Pseudomonas aeruginosa | 1.25% | 2.5% |
| Salmonella enterica serovar typhimurium | 0.04% | 0.04% |
| Staphylococcus aureus | 0.31% | 0.16% |
| Staphylococcus epidermis | 0.31% | 0.16% |
| Streptococcus mutans | 0.31% | 2.5% |
| Streptococcus oralis | 0.635% | 0.16% |
| Veillonella dispar | 1.25% | 1.25% |
| Vibrio splendidus | 2.5% | 2.5% |
| Vibrio cholerae | 0.08% | 0.08% |
| Yersinia enterolytica | 0.08% | 0.04% |
| Aspergillus niger | 0.31% | 0.635% |
| Candida albicans | 1.25% | 1.25% |
| Malassesia furfur | 1.25% | 0.635% |

None of the tested oils presented antibacterial activity against Clostridium perfringens. Both tested oils have similar antibacterial activities against Bacillus cereus, Salmonella enterica ser typhymurium, Veillonella dispar, Vibrio splendidus and Vibrio cholera. ISO TTO has a better antibacterial activity than exemplary TTO derivatives against 3 bacterial strains: Fusobacterium nucleatum, Pseudomonas aeruginosa and Streptococcus mutans.

By contrast, exemplary TTO derivatives showed stronger antibacterial activity than ISO TTO against 10 bacterial strains: Actinomyces naeslundi, Bacillus atrophaeus, Escherichia coli, Legionella pneumophila, Listeria monocytogenes, Propionibacterium acnes, Staphylococcus aureus, Staphylococcus epidermidis, Streptococcus oralis and Yersinia enterolytica. This superior effect of exemplary TTO derivatives on ISO TTO was unexpected since it is well documented that molecules such as α-pinene, 1,8-cineole, α-terpinene, γ-terpinene, p-cymene are strong antibacterial molecules, and are absent from exemplary TTO derivatives but present in ISO TTO.

Concerning the three fungi, ISO TTO and exemplary TTO derivatives have similar antifungal activities against Candida albicans. ISO TTO is more effective than exemplary TTO derivative against Aspergillus niger, but less against Malassesia furfur. Thus, exemplary TTO derivatives are at least as effective as TTO concerning the antibacterial and antifungal activities.

Exemplary TTO derivatives could thus be incorporated at 1.25% final concentration in intimate hygiene products (soap, gel, vaginal capsules, vaginal creams) to prevent urinary tract infections, or prevent or cure vaginosis, at 1.25% final concentration in anti-acne compositions (lotion, soap, cream) to prevent or cure acne, at 1.25% final concentration in oral care products (toothpaste, gum, mouthwash) to prevent or cure mouth infections, candidosis, caries, gingivitis, periodontis), at 0.31% final concentration as a preservative for ingredient preservation, food or cosmetics, at 0.16% to 2.5% final concentration in solutions for disinfection (gel, spray), the lower concentrations being efficient against Salmonella, Yersinia, Bacillus strains, the higher concentrations being efficient against Vibrio and Legionella. This also includes hygiene compositions such as antibacterial and antifungal compositions for hygiene such as creams and balms for podologists.

### Example 6

### Method of Treating Viral Infections by Administering Effective Doses of Exemplary TTO Derivatives

Arthropod-borne flaviviruses including, but not limited to, dengue (DEN), Japanese encephalitis (JE), West Nile (WN) and yellow fever (YF) viruses ,cause extensive morbidity and mortality worldwide. Among flaviviruses the dengue viruses (DENVs), causal agents of dengue fever (DF) arguably have the most significant numerical impact on the public health. Dengue viruses are enveloped, positive-sense RNA viruses in the Flaviviridae family, flavivirus genus, and are transmitted to humans by infected mosquitoes. There are four antigenically-related virus serotypes, DENV types 1, 2, 3 and 4, which can be differentiated serologically by virus neutralization tests. Although immunity to the infecting serotype(s) is believed to be life-long, cross-serotype protection is usually only short-lived due to waning antibody titers, and primary infection may predispose to more severe disease upon later secondary infection with a different serotype.

The disease burden from dengue infection is escalating worldwide, especially in tropical and subtropical regions due to factors such as global climate change, increased population mobility, and inadequate vector control. It is estimated that over 100 million people are infected each year resulting in almost 50,000 deaths (reviewed by Henchal and Putnak, 1990; Monath, 1990; Weaver and Reisen, 2010). There are as yet no approved vaccines or prophylactic drugs for dengue. The leading tetravalent live-attenuated dengue vaccine candidates may require up to nine months or more to induce protective immunity; as such they may be poorly suited for many high risk groups including travelers to endemic areas and the military. Therefore, much effort has been devoted to the evaluation of potential dengue antiviral drugs, both for disease prevention and treatment. Unfortunately, research on dengue vaccines and antivirals has been hampered by the lack of good animal disease models. Recently, it has been shown that immune-compromised mice reconstituted with human hematopoietic stem cells can be infected with dengue virus and that these infections mimic dengue virus tropism resulting in a full spectrum of dengue-like disease (Mota and Rico-Hesse, 2011). However, these specialized mouse strains are very expensive and difficult to produce and maintain. The non-human primate (e.g., rhesus macaque) infection model is currently the most widely accepted animal model for the evaluation of dengue vaccines and antiviral drug candidates. In this model infection is typically asymptomatic and the duration, onset, and magnitude of viremia are used as disease surrogates (Putnak et al., 2005).

Although several candidate dengue antivirals have been tested, none have ideal drug profiles and long-term safety and efficacy are major concerns. One compound isolated from an essential oil of Melaleuca alternifolia (IMA) has been studied as a possible alternative to more conventional drugs. IMA extract, whose common name is TTO, is approved for use as an herbal medicine in Australia and is sold in the United States as a health supplement.

An exemplary TTO derivative described herein, designated “FJ1,” or a placebo was administered orally to rhesus macaques. After one week of daily dosing the drug was stopped and the animals were challenged with live, non-attenuated DENV2 and circulating infectious virus (serum viremia) and viral RNA were measured. An antiviral effect was assessed after virus challenge by the measurement of circulating live virus (viremia), viral RNA, and virus neutralizing antibody responses. The animals that received FJ1exhibited a nearly 50% reduction in the duration of viremia. Although peak viral RNA titers were not significantly reduced compared to placebo controls, the total RNA burden in FJ1treated animals declined more rapidly and there were lower post-challenge virus neutralizing antibody titers, consistent with reduced viral replication. FJ1 was well tolerated with no adverse effects observed during the exemplary TTO derivative dosing period or after follow-up for two months. In vitro studies demonstrated a direct inhibitory effect for FJ1on DENV2 replication in Vero cells under conditions of low input virus multiplicity designed to mimic natural infection. Taken together, the results indicate that FJ1may be a candidate antiviral drug for clinical development with an indication to reduce infection and prevent disease.

### Materials and Methods

Rhesus macaques. Ten (10) adult rhesus macaques (Indian strain), approximately 7 to 12 Kg in weight, both males and females, apparently healthy with no history of past flavivirus exposure, were used. Prior to testing, animals were given physical exams and were tested and screened negative for antibodies to multiple flaviviruses: DENV 1, 2, 3 and 4, Japanese encephalitis, WN and Saint Louis encephalitis (SLE) virus by viral plaque reduction neutralization test (PRNT) and/or hemagglutination inhibition (HAI) assay. Animals were also tested and screened negative for TB reactivity, antibodies to SIV, STLV, simian herpes B virus and SRV by cell culture or PCR assay. The animals were then randomly divided into two treatment groups, five that received the candidate FJ1 and five rice flour placebo controls.

FJ1 Antiviral drug. Compound FJ1, a partially purified derivative of TTO, was provided by study sponsor, A.M.S. Biotek LLC. For use in the animal study, compound FJ1 was provided in capsule form with each capsule containing 370 mg of active ingredient in a rice flour binder; capsules containing only rice flour were provided as a placebo control. For the in vitro experiments compound FJ1 was provided as a 10% solution solubilized in 6% polysorbate 60; 6% polysorbate 60 alone was provided as a control. All formulations were stored at room temperature.

### Antiviral Drug and Placebo Control Preparations and Administration.

Individual doses for each animal were prepared by emulsifying the contents of an appropriate number of capsules in 3 to 5 ml of Ensure® brand nutrient drink, which was then administered to lightly Ketamine-sedated animals by gastric lavage, followed by 2-3 ml of normal saline to flush the feeding tube. Compound FJ1 at a dosage of 80-100 mg/Kg or the placebo was administered daily for seven days (study days 0-6) at approximately the same time each day. The dosing was then stopped and the animals were challenged with dengue virus type 2 on study day 7.

Virus challenge. Virus challenge was performed by subcutaneous inoculation of 5 log10 Vero cell plaque-forming units of non-attenuated dengue type 2, strain S16803 virus. The infectivity titer of the challenge virus was confirmed by plaque assay on Vero cell monolayers. Blood was collected daily from the animals for 14 days after challenge.

Dengue/flavivirus serology. Sera processed from coagulated whole blood were used for measuring pre- and post-challenge dengue type 2 virus neutralizing antibodies by a plaque reduction neutralization test (PRNT50) on Vero cells in six-well plates (Putnak et al., 2008). Prior to the study animals were screened by PRNT50 and/or hemagglutination inhibition (HAI) test (Clarke and Casals, 1958) for antibodies to DENV 1, 2, 3 and 4, JE, SLE and WN flaviviruses and only antibody-negative animals were used in the study.

Dengue viremia assay. Blood obtained daily for 14 days after virus challenge was coagulated, processed to serum and used to measure serum viremia by a fourteen-day dengue virus amplification assay on Vero cells followed by direct plaque titration assay. Briefly 0.1 ml of each serum was inoculated onto Vero cells in T25 flask; cells were incubated at 35 C for seven days, re-fed with fresh medium and the culture supernatants harvested on day 14. Virus was detected by plaque assay of culture supernatants on Vero cells in six-well plates.

Quantitative PCR. Viral RNA extraction. Viral RNA for Quantitative RT-PCR (Q-RT-PCR) was extracted from 200 μl of serum by Qiagen Viral RNA Kit according to the manufacturer's instructions.

Quantitative RT-PCR. The primers and fluorogenic probes used in Q-RT-PCR have been previous described in Sadon et al. 1998, Journal of Virological methods. Dengue 2 was detected with a reaction mixture containing 5 μl of RNA sample, 10 pmol dengue 2 primers and 5 pmol dengue 2 probe, 25 μl of Quantitect probe RT-PCR master mix (QIAGEN), 0.5 μl of Quantitect RT-Mix (QIAGEN), and 4 U of RNase inhibitor (Promega) in 50-μl total volume. The one step RT-PCR consisting of a 30-min RT step at 50° C. and 15 min of Taq polymerase activation at 95° C., followed by 40 cycles of PCR at 95° C. for 15 sec, and 60° C. for 1 min was performed in the ABI Prism 7900 sequence detector. A sample was defined empirically as positive if the cycle number (C_{T} value) was <40, based on background cross-reactivity of the primers and probes in nontemplate control reactions. The RNA copies number was obtained from standard curves of 10-fold dilutions, and 5-fold dilutions of In vitro RNA transcripts of DENV-2 with known copies numbers. The limit of detection for this assay is 5 genomic copies per rxn.

Complete Blood Count (CBC) and Serum Chemistry. A CBC with differential including platelet count and a serum chemistry panel (Chem-20) were performed by an accredited laboratory in the Division of Pathology, WRAIR, according to previously validated procedures. Results were interpreted by a Veterinary Pathologist.

Vero cells. The Vero-81 cells used in this study were obtained from the American Type Culture Collection (ATCC, Vienna, Va.) and a working cell bank was established at Passage-128. Prior to subculture, cells were propagated in tissue culture treated (T175 cm2) flasks in complete Eagles Minimal Essential Medium (EMEM) with 10% fetal calf serum (FCS), 1× L-glutamine, 1× non-essential amino acids, and 1× Pen/strep, in a 35 C, 5% CO2 incubator.

Dengue virus type-2 (DENV2) strain S16803. The DENV2 virus working stock used in the viral infectivity inhibition assay (see below) was prepared by infection of Vero cells grown in a T175 cm² flask with DENV2, strain S16803, and incubation at 35 C in a 5% CO2 incubator. The infected culture supernatant fluid was collected after seven days of incubation, clarified it by centrifugation, aliquoted, and stored at -80 C.

Vero cell cytotoxicy assays. Compound FJ1 solubilized in 6% polysorbate 60 was compared to an excipient control (polysorbate 60 alone) and a no treatment control for cytotoxicity in Vero cell monolayers subcultured in 96-well plates. Serial two-fold dilution of FJ1 or polysorbate 60 excipient ranging from 1% to 0.002% were added to cell monolayers and incubated at 35 C, 5% CO2. The cells were examined over four days in culture by light microscopy to detect morphological changes which might indicate cytotoxic effects, including cell rounding, detachment from the monolayer, membrane changes, and granulation compared with untreated control cells.

In vitro viral infectivity inhibition assay. The effect of FJ1 on DENV2 replication in Vero cells was determined by an indirect immunofluorescence assay. For this assay the DENV2 stock prepared as described above was initially diluted 1:100 in EMEM/2% to an estimated titer of 10⁴ plaque forming units (PFU), then serially 10-fold down to 1 plaque PFU/ml. A 100 μl aliquot of each virus dilution was incubated for 30 min with 100 μl of Compound FJ1 in polysorbate 60 or polysorbate 60 alone. After incubation the mixtures were transferred to Vero cell monolayers subcultured in 48-well plates. After 48 hours in culture (35 C, 5% CO2) the cells were removed from the wells by trypsin, transferred to the wells of a glass microscope 6-well slide, air dried and fixed with ice cold acetone. Fixed cells were reacted with monoclonal antibodies 3H5 and 4G2 to the envelope (E) protein or monoclonal antibody 7E11 to the NS1 protein followed by goat anti-mouse secondary antibody conjugated to FITC. Stained cells were visualized by fluorescence microscopy and photographed.

Statistical Analysis. Statistical analysis, where indicated, was performed by the Wilcoxon Two-Sample Exact Test implemented on a microcomputer running SAS analytic software (SAS Institute, version 9.3, Cary, N.C.).

### Results

Safety and Efficacy of FJ1 in rhesus macaques. This was a placebo-controlled, non-blinded study to test the effect of compound FJ1, an exemplary TTO derivative, on dengue type 2-virus infection in rhesus macaques. As shown in Table 1 (all Tables below), groups of five animals each received either FJ1 (80-100 mg/Kg) or a rice flour placebo emulsified in Ensure® nutrient drink administered by gastric lavage. The schedule of drug administration was once daily for 7 days (see Table 2). Rarely, an animal received an incomplete dose (although not less than 50% of a full dose) because of the thickness of the emulsion and difficulty in transiting the gastric feeding tube. Daily assessments of the animals' general well-being (e.g., grooming, eating, etc.), body weight and temperature, heart and respiration rates, as well as the results of CBC and serum chemistries performed on days 0 and 7 were unremarkable in drug-treated animals, with no significant variations from the animals' pre-study baseline measurements (see Supplementary Data Excel Spreadsheet), except that one animal (DA4D) in the placebo control group exhibited an adverse reaction to the daily Ketamine anesthesia. This was accompanied over the course of the study by progressive weight loss and behavioral problems including self-mutilation possibly induced by stress, which necessitated humane euthanasia of the animal on study day 16.

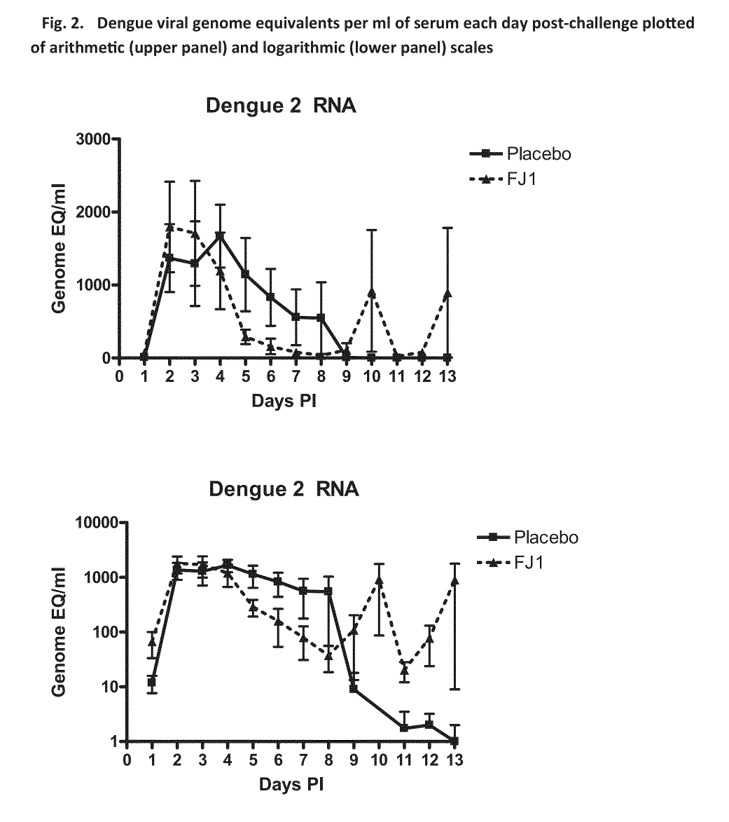

On study day 7, at the end of the dosing period the dosing was stopped and all ten animals were challenged with live dengue type 2 virus. After challenge, small quantities of blood were collected daily for 14 days for viremia and quantitative viral RNA determinations. As shown in Table 3, the group that received FJ1 had a total of 15 days of viremia (range 2 to 4 days), compared with at least 26 days of viremia (range 3 to ≧8 days) in the placebo controls. This difference was significant (P=0.03). It should be noted that subject DA4D in the placebo group was still viremic at the time of its euthanasia, so that a viremia endpoint could not be determined. If the viremia data for DA4D were to be excluded from the analysis then the difference between the drug and placebo groups would no longer be significant (P=0.07). In order to measure the magnitude or intensity of the viremia, virus-positive sera were titered by direct plaque assay in Vero cells. Virus titers measured by plaque assay were highest for DA4D, ranging from 25 to 325 pfu/ml; whereas all other animals exhibited much lower titers of virus (maximum of 75 pfu/ml) in their sera (Table 3). The sera were also tested independently by a quantitative RT-PCR (qRT-PCR) assay to measure circulating viral RNA, reported as genome equivalents. The qRT-PCR assay results for individual animals are shown in Table 3A and the group means are presented graphically in FIG. 2. It can be seen that the amount of circulating viral RNA (presented on both an arithmetic and log scale) declined more rapidly in FJ1 treated animals compared to placebo controls. It should be noted that since the qRT-PCR assay cannot distinguish between infectious and non-infectious virus the RNA measurements will also include immature DENV particles released from infected cells and virus inactivated by either the drug or the host immune response. For DENVs the ratio of infectious to non-infectious particles is estimated to be in the range 1:100 to 1:1,000. Consistent with this, the results from qRT-PCR assay clearly showed positive results in more samples and higher titers than the viremia plaque assays.

Approximately one month after the resolution of viremia, all animals were tested for neutralizing antibodies against the challenge virus by PRNT assay (Table 4). The virus neutralizing antibody titers were found to be two-fold to three-fold higher in placebo controls, suggestive of more virus replication in those animals compared to animals that had received FJ1. This difference was significant (P=0.0079).

Cytotoxicity of FJ1 for Vero cells. As shown in Table 5, cytotoxicity testing on Vero cell monolayers demonstrated that compound FJ1 was not toxic until exposure to concentrations of 0.0.015%% or higher. At concentrations of 0.03% or higher morphological changes were observed in all cells after four days in culture including rounding, membrane changes, granulation, and detachment from the monolayer, compared with untreated control cultures. At a FJ1 concentration of 0.015% a small proportion of cells appeared to remain healthy; this concentration was defined as the sub-toxic dose. Cells treated with concentrations of FJ1 of 0.007% or less exhibited no changes compared to the untreated control. This concentration was defined as the non-toxic dose. The polysorbate 60 solubilizer by itself was found to be less toxic, with a sub-toxic dose of 0.125% and non-toxic dose of 0.06%. Cytotoxicity did not progress after three additional days in culture in the presence of FJ1 or polysorbate 60 (data not shown).

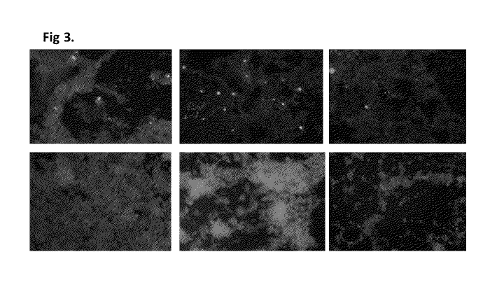

Effect of FJ1 on DENV2 replication in Vero cells. FJ1 dissolved in polysorbate 60 was assayed for effect on DENV2 viral replication in Vero cells at sub-toxic and non-toxic concentrations (see Methods) using virus concentrations ranging from approximately 10⁴ PFU/ml down to 1 PFU/ml. Several conditions were tested including pre-incubation of virus and drug and pre-treatment of cells with drug prior to infection. Infected cells were detected by staining with monoclonal antibodies against virion envelope (E) protein (3H5 and 4G2) or NS1 protein (7E11) in an indirect immunofluorescence assay (IFA). The results demonstrated that 0.0035% FJ1 pre-incubated with 1 PFU/ml of DENV2 virus resulted in a reduction in infected cells compared to control (FIG. 3: DENV-2 (1 PFU/ml) was preincubated with FJ1 (0.0035%) in EMEM/2% FCS or with EMEM/2% FCS alone (control) for 30 minutes at 35° C. (in triplicate) then inoculated onto Vero cells in 48-well plates. Cells were incubated for 48 hours, then assayed by indirect immunofluorescence using anti-NS1 7E11 mAb. Top row: virus without FJ1; bottom row: virus with FJ1). However, pre-treatment of cells with drug prior to addition of virus did not lead to an observable reduction in infected cells (data not shown).

The results of this work demonstrated that compound FJ1, when administered to rhesus macaques in daily doses of 80-100 mg/Kg for one week prior to challenge with dengue type-2 virus, was effective at reducing the duration of circulating live virus (viremia) by approximately 50% compared to a placebo. The peak amount of circulating viral RNA was not significantly different between groups in the first 4 days post-challenge, which suggests that FJ1 pretreatment before the challenge did not affect initial viral infection and viral replication in host cells after the challenge. However, RNA titers and viral titers appeared to decline more rapidly in the FJ1-treated animals compared with placebo controls starting from Day 5. As demonstrated by Witarto (2007), dengue viruses exposed to IMA in vitro undergo a change from quasi-spherical to an un-ordered shape. It can be speculated that interactions between FJ1 or other IMA derivatives and host cell or possibly viral membranes during drug uptake may trigger the release of more abnormal forms of the virion (which are detectable by qRT-PCR but not by cell culture assay), thereby resulting in stimulated viral clearance. Alternatively, the drug may act to stimulate early T-cell responses that effect more rapid clearance of virus-infected cells. Animals that received compound FJ1 also had significantly lower dengue virus neutralizing titers measured six weeks after challenge, consistent with reduced levels of virus replication in those animals.

Compound FJ1 also appeared to be well tolerated, with no adverse effects observed in the drug-treated group during the seven-day dosing period or for two months thereafter. Only one animal, DA4D, in the placebo group exhibited any adverse reactions during the study. These occurred after Ketamine anesthesia for the daily blood collections, and included progressive weight loss and behavioral problems, the severity of which necessitated the animal's euthanasia on study day 16. Prior to that DA4D was apparently healthy.

The mechanism of action also remains largely unknown. Although disruption of virion envelope in the presence of IMA has been observed in vitro (Witarto, 2007) it is not known if this also occurs in vivo where physiologic concentrations of the drug would be much lower. One study reported an early inhibitory effect of IMA on influenza virus replication, possibly at the level of virus uncoating (Garozzo et al., 2011), which supports an interaction with viral and/or host cell membranes. In the present study inhibition of DENV2 replication in Vero cells by FJ1 was observed only under conditions where a very low concentration of input virus was pre-incubated with drug before being added to the Vero cells. While not definitive, these results are suggestive of a direct effect of FJ1 on the dengue virion, possibly by interaction with the viral membrane. It is also possible that certain Melaleuca alternifolia compounds, as antioxidants (Kim et al., 2004), have beneficial effects beyond a putative direct anti-viral effect such as the reduction of host inflammatory responses (Hart et al., 2000; Caldefie-Chezet, 2004, 2006; Golab and Skwarlo-Sonta, 2007) or stimulation of cell mediated immune responses, which may also play a role in reducing the severity of dengue disease. Compound FJ1 appears to have a distinct advantage over conventional antivirals by virtue of its excellent safety profile, which could allow for its long-term prophylactic use against dengue.

In summary compound, FJ1, an exemplary TTO derivative, was demonstrated in this work to be safe and effective at reducing DENV2 infection in a rhesus macaque model. When pre-incubated with a low concentration of DENV2 virus in vitro FJ1 appeared to inhibit infection of Vero cells.

Without limitation to only those disclosed herein and without disclaimer of other embodiments, compositions comprising some embodiments are administered and dosed in accordance with good medical practice, taking into account the clinical condition of the individual patient, the site and method of administration, scheduling of administration, patient age, sex, body weight and other factors known to medical practitioners. The “pharmaceutically effective amount” for purposes herein is thus determined by such considerations as are known in the art. The amount must be effective to achieve improvement, including but not limited to, decreased indicators of disease, decreased frequency or severity of disease, or improvement or elimination of symptoms and other indicators as are selected as appropriate measures by those skilled in the art.

Administration can be in various ways. It can be administered alone or as an active ingredient in combination with pharmaceutically acceptable carriers, diluents, adjuvants and vehicles. Administration can be oral, subcutaneous or parenteral including intravenous, intraarterial, intramuscular, intraperitoneal, and intranasal administration as well as intrathecal and infusion techniques, or by local administration or direct inoculation to the site of disease or pathological condition. Implants of the compounds are also useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the experimental animals exemplified herein which treatment has a length proportional to the length of the disease process and drug effectiveness. The doses may be single doses or multiple doses over periods of time. The treatment generally has a length proportional to the length of the disease process and drug effectiveness and the patient species being treated.

When administering some embodiments parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

When necessary, proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for selected embodiments. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. Any vehicle, diluent, or additive used should be compatible with the selected embodiment.

Sterile injectable solutions can be prepared by incorporating the desired embodiment in the required amount of the appropriate solvent with various of the other ingredients, as desired.

A pharmacological formulation comprising some embodiments can be administered to the patient in an injectable formulation containing any compatible carrier, such as various vehicle, adjuvants, additives, and diluents; or the embodiments can be administered parenterally to the patient in the form of slow-release subcutaneous implants or targeted delivery systems. Many other such implants, delivery systems, and modules are well known to those skilled in the art.

Without limitation to only those disclosed herein and without disclaimer of other embodiments, compositions comprising some embodiments may be in any suitable form and for internal or external use. Preparations for internal use include powders, tablets, dispersible granules capsules, solutions, suspensions, and emulsions suitable for oral ingestion or injection.

Compositions may also find use as a topical anti-microbial and/or anti-parasitic agent. Examples of such applications include antiseptic scrubs or washes and flea and lice shampoos, spray, plunge dips and pur-on formulations.

Topical compositions may also be administered in the form of wound dressings, transdermal patches and the like.

Compositions may be applied in the form of an aerosol. Such formulations may be used for the treatment of lung infections. Compositions may also be used in the form of a fumigant for the purposes of controlling infectious agents in an animal enclosure such as a dairy, stable, pen or the like. A particular application is the fumigation or fogging of bee hives to control or treat mite infestations.

In some embodiments, without limitation to only those disclosed herein and without disclaimer of other embodiments, effective dosing may be determined in accordance with the disease, the level of infection encountered, routes, frequency, and other considerations of prophylaxis, and other considerations and techniques known or available to the skilled artisan and applied consistent with good scientific or medical standards and practice.

Without limitations, some embodiments may be used in conjunction with any form of plant or animal, including but not limited to, humans and other mammals.

All attachments, tables, and cited references are hereby incorporated by reference in their entireties as though fully set forth herein.

It will be appreciated that various changes or modifications may be made to embodiments as described and claimed herein without departing from the spirit and scope thereof. While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements.

The foregoing embodiments are illustrative and not restrictive, and no single feature or element is essential to all possible combinations that may be claimed in this or a later application. Many embodiments and applications other than the examples provided would be apparent to those of skill in the art upon reading the above description. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future embodiments. In sum, it should be understood that the invention is capable of modification and variation and is limited only by the following claims.

All terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. Where the claims recite “a” or “a first” element of the equivalent thereof, such claims should be understood to include incorporation of one or more such elements, neither requiring nor excluding two or more such elements.

### Tea Tree oil Derivative

TABLE 1
Treatment Groups

| Animal ID | Weight (Kg) | Treatment (once per day) |
|---|---|---|
| CI75 | 11.12 | Placebo - Rice flour in 3-5 ml Ensure ® |
| RQ703C | 7.89 | Placebo - Rice flour in 3-5 ml Ensure |
| DA4D | 8.81 | Placebo - Rice flour in 3-5 ml Ensure |
| 03D113 | 10.31 | Placebo - Rice flour in 3-5 ml Ensure |
| M032R | 10.98 | Placebo - Rice flour in 3-5 ml Ensure |
| DB3Y | 11.48 | FJ1 - 910 mg in 3-5 ml Ensure |
| DA4J | 8.85 | FJ1 - 710 mg in 3-5 ml Ensure |
| DA1G | 8.58 | FJ1 - 690 mg in 3-5 ml Ensure |
| DB4R | 9.14 | FJ1 - 730 mg in 3-5 ml Ensure |
| DB3C | 7.64 | FJ1 - 610 mg in 3-5 ml Ensure |

TABLE 2
Schedule of Events

| | | Urine | Blood |
|---|---|---|---|
| Study Day | Events | collection | collection |
| −7 to −14 | 1) Baseline CBC, Chem, Serology, | X | X |
| | Urinalysis. | | |
| 0 | 1) Observation/Weight/TPR. | X | X |
| | 2) Baseline blood and urine for | | |
| | Pharmacokinetics (PK). | | |
| | 3) Drug administration. | | |
| 1 | 1) Observation/Weight/TPR. | | |
| | 2) Drug administration. | | |
| 2 | 1) Observation/Weight/TPR. | | |
| | 2) Drug administration. | | |
| 3 | 1) Observation/Weight/TPR. | X | |
| | 2) Blood and urine for PK. | | |
| | 3) Drug administration. | | |
| 4 | 1) Observation/Weight/TPR. | | |
| | 2) Drug administration. | | |
| 5 | 1) Observation/Weight/TPR. | | |
| | 2) Drug administration. | | |
| 6 | 1) Observation/Weight/TPR. | | |
| | 2) Drug administration. | | |
| 7 | 1) Observation/Weight/TPR | X | X |
| | 2) CBC, Chem, Urinalysis, PK | | |
| | 3) Baseline (pre-challenge) | | |
| | viremia | | |
| | 4) Virus challenge | | |
| 8 to 20 | 1) Observation/Weight/TPR | | X |
| | 2) Bleed for viremia | | |
| 28 | 1) Observation/Weight/TPR | X | X |
| | 2) CBC, Chem, Urinalysis | | |
| 48 | 1) Observation/Weight/TPR | X | X |
| | 2) CBC, Chem, Urinalysis | | |
| | 3) Dengue serology | | |
| 72 | 1) Final Observation/Weight/TPR | X | X |
| | 2) Final CBC, Chem, Urinalysis | | |

TABLE 3
Viremia in Recipients of FJ1 and Placebo after Challenge with Dengue Type-2 Virus

| Animal | Treatment | Presence of viremia and virus titer (Pfu/ml) on each day post-DEN2 virus challenge* | | | | | | | | | | | | | |
|---|---|---|---|---|---|---|---|---|---|---|---|---|---|---|---|
| ID | Group | 1 | 2 | 3 | 4 | 5 | 6 | 7 | 8 | 9 | 10 | 11 | 12 | 13 | Total |
| DB3Y | Drug FJ1 | 0 | +(<25) | +(<25) | +(<25) | 0 | 0 | 0 | 0 | 0 | 0 | 0 | 0 | 0 | 3 |
| DA4J | Drug FJ1 | 0 | +(50) | +(50) | +(50) | +(<25) | 0 | 0 | 0 | 0 | 0 | 0 | 0 | 0 | 4 |
| DA1G | Drug FJ1 | 0 | +(50) | +(<25) | 0 | 0 | 0 | 0 | 0 | 0 | 0 | 0 | 0 | 0 | 2 |
| DB4R | Drug FJ1 | 0 | +(<25) | +(<25) | +(75) | +(25) | 0 | 0 | 0 | 0 | 0 | 0 | 0 | 0 | 4 |
| DB3C | Drug FJ1 | 0 | +(50) | +(<25) | 0 | 0 | 0 | 0 | 0 | 0 | 0 | 0 | 0 | 0 | 2 |
| FJ1 Group cumulative days of viremia | | | | | | | | | | | | | | | 15 |
| C175 | Placebo | 0 | 0 | +(<25) | 0 | +(<25) | +(<25) | 0 | 0 | 0 | 0 | 0 | 0 | 0 | 3 |
| RQ703C | Placebo | +(<25) | +(75) | +(75) | +(25) | +(25) | +(<25) | 0 | 0 | 0 | 0 | 0 | 0 | 0 | 6 |
| DA4D | Placebo | 0 | +(25) | +(50) | +(275) | +(125) | +(325) | +(300) | +(50) | +(25) | Nd | Nd | Nd | Nd | 8 |
| 03D113 | Placebo | 0 | +(50) | +(50) | +(<25) | +(50) | +(<25) | 0 | 0 | 0 | 0 | 0 | 0 | 0 | 5 |
| M032R | Placebo | 0 | +(25) | +(75) | +(50) | +(<25) | 0 | 0 | 0 | 0 | 0 | 0 | 0 | 0 | 4 |

| | |
|---|---|
| Placebo Group cumulative days of viremia | 26 |

*Determined by 14 day amplification of Vero-81 cells followed by Vero cell plaque assay
Nd, not determined (animal euthanized on study day 9)

TABLE 3A
Viral RNA in Sera of Recipients of FJ1 and Placebo after Challenge with Dengue Type-2 Virus

| Animal | Treatment | Dengue genome equivalents per ml of serum on each day post-DEN2 virus challenge* | | | | | | | | | | | | | |
|---|---|---|---|---|---|---|---|---|---|---|---|---|---|---|---|
| ID | Group | 1 | 2 | 3 | 4 | 5 | 6 | 7 | 8 | 9 | 10 | 11 | 12 | 13 | Total |
| DB3Y | Drug FJ1 | 54 | 363 | 6 | 151 | 174 | 22 | 29 | 41 | 49 | 337 | 3 | 77 | 16 | 1322 |
| DA4J | Drug FJ1 | 77 | 3600 | 2891 | 1597 | 280 | 47 | 15 | 12 | 0 | 0 | 0 | 0 | 0 | 8519 |
| DA1G | Drug FJ1 | 189 | 2385 | 504 | 1151 | 372 | 572 | 270 | 108 | 488 | 4246 | 41 | 285 | 4443 | 15054 |
| DB4R | Drug FJ1 | 15 | 424 | 3823 | 2954 | 605 | 144 | 20 | 17 | 6 | 9 | 30 | 26 | 20 | 8093 |
| DB3C | Drug FJ1 | 0 | 2208 | 1319 | 116 | 20 | 13 | 63 | 8 | 0 | 11 | 27 | 0 | 0 | 3785 |
| FJ1 Group cumulative days of RNAemia | | | | | | | | | | | | | | | 56 |
| C175 | Placebo | 11 | 222 | 366 | 351 | 501 | 864 | 223 | 12 | 0 | 0 | 7 | 0 | 0 | 2557 |
| RQ703C | Placebo | 11 | 2731 | 1324 | 1166 | 694 | 297 | 119 | 24 | 0 | 0 | 0 | 5 | 0 | 6371 |
| DA4D | Placebo | 0 | 953 | 53 | 2714 | 3144 | 2327 | 2085 | 2507 | nd | Nd | Nd | nd | nd | 13783 |
| 03D113 | Placebo | 11 | 770 | 1350 | 2454 | 742 | 437 | 179 | 42 | 0 | 0 | 0 | 3 | 0 | 5988 |
| M032R | Placebo | 26 | 2165 | 3375 | 1665 | 636 | 229 | 187 | 151 | 36 | 0 | 0 | 0 | 4 | 8474 |

| | |
|---|---|
| Placebo Group cumulative days of RNAemia | 42 |

*Determined by qRT-PCR
Nd, not determined (animal euthanized on study day 9)

TABLE 4
Dengue virus-Neutralizing Antibody Titers six-weeks
Post-Challenge in Recipients of FJ1 and Placebo

| Animal | Treatment | Neutralizing Antibody (PRNT50) Titers | |
|---|---|---|---|
| ID | Group | Day 0 | Day 48 |
| DB3Y | Drug FJ1 | <10 | 250 |
| DA4J | Drug FJ1 | <10 | 625 |
| DA1G | Drug FJ1 | <10 | 233 |
| DB4R | Drug FJ1 | <10 | 265 |
| DB3C | Drug FJ1 | <10 | 250 |
| Drug FJ1 Group Mean PRNT50 Titer | | | 325 |
| C175 | Placebo | <10 | 633 |
| RQ703C | Placebo | <10 | 920 |
| DA4D | Placebo | <10 | Nd |
| 03D113 | Placebo | <10 | 762 |
| M032R | Placebo | <10 | 934 |

| | |
|---|---|
| Placebo Group Mean PRNT50 Titer | 812 |

Nd, Not determined

TABLE 5
Cytotoxic Effect of Compound FJ1 and polysorbate 60 (PS-60) for Vero Cells

| % FJ1 | 1.0 | 0.5 | 0.25 | 0.125 | 0.06 | 0.015 | 0.007 | 0.0035 | 0.00175 |
|---|---|---|---|---|---|---|---|---|---|
| Observed | All cells | All cells | Membrane | Membrane | Intracellular | Healthy | Healthy | Healthy | Healthy |
| CPE | destroyed | destroyed | dissolved | dissolved | granulation | (10%) | (100%) | (100%) | (100%) |
| % PS-60 | 1.0 | 0.5 | 0.25 | 0.125 | 0.06 | 0.015 | 0.007 | 0.0035 | 0.00175 |
| Observed | All cells | All cells | All cells | Cells | Healthy | Healthy | Healthy | Healthy | Healthy |
| CPE | destroyed | destroyed | destroyed | rounded/ | (100%) | (100%) | (100%) | (100%) | (100%) |
| | | | | dead | | | | | |

What is claimed is:

1. A tea tree oil derivative composition, comprising:
a terpinolene that is below 1.0%;
a-terpinene that is below 0.5%;
g-terpinene that is below 3.0%;
p-cymene that is below 0.5%; and
at least one of a monoterpenic alcohol that is more than 60.0%, a terpinen-4-ol that is more than 60.0%; and a a-terpineol that is more than 4.0%.

2. A method for the treatment or prophylaxis of viral infection in a mammal, comprising the step of administering to said mammal a pharmaceutically effective amount of the composition of claim 1.

3. The method of claim 2, wherein the viral infection is associated with dengue virus.

4. An antimicrobial composition for ingestion or application to humans comprised of the composition of claim 1.

5. A disinfecting composition comprised of the composition of claim 1.

6. A method of eliminating toxic and irritable molecules from tea tree oil, comprising:
enriching a tea tree oil substrate in a beneficial molecule;
depleting the tea tree oil substrate of at least one of a toxic molecule and an irritable molecule; and
providing the tea tree oil substrate that has a α-pinene that is below 0.05%.

7. The method of claim 6, wherein the beneficial molecule is at least one of a sesquiterpene and an alcohol.

8. The method of claim 6, wherein the at least one of the toxic molecule and the irritable molecule is a monoterpene.

9. The method of claim 6, further comprising:
providing the tea tree oil substrate that has a terpinolene that is below 1.0%;
providing the tea tree oil substrate that has a a-terpinene that is below 0.5%;
providing the tea tree oil substrate that has a g-terpinene that is below 2.5%;
providing the tea tree oil substrate that has a p-cymene that is below 0.5%;
and
providing the tea tree oil substrate that has a total amount of monoterpenes below 5.0%.

10. The method of claim 6, further comprising:
providing the tea tree oil substrate that has a monoterpenic alcohol that is more than 60.0%.

11. The method of claim 6, further comprising:
providing the tea tree oil substrate that has a terpinene-4-ol that is more than 60.0%; and
providing the tea tree oil substrate that has a α-terpineol that is more than 4.0%.
